(19) United States
(12) Patent Application Publication
(10) Pub. No.: US 2006/0128990 A1
(43) Pub. Date: Jun. 15, 2006

(54) Non-corrosive amphoteric surfactants and method of well treatment

(76) Inventors: Stephen T. Davis, Missouri City, TX (US); Vaithilingam Panchalingam, Friendswood, TX (US)

Correspondence Address: STREETS & STEELE, 13831 NORTHWEST FREEWAY, SUITE 355, HOUSTON, TX, 77040, US

(21) Appl. No.: 10/518,617

(22) PCT Filed: Jun. 26, 2003
(86) PCT No.: PCT/US03/20597

§ 371 Date: Dec. 5, 2005

Related U.S. Application Data
(60) Provisional application No. 60/391,563, filed on Jun. 26, 2002.

(51) Int. Cl. C07C 227/10

(52) U.S. Cl. 562/553

(57) ABSTRACT

A chloride free amphoteric surfactant obtained by reacting an amine with a carbonyl compound, the amphoteric surfactant being particularly useful as a foamer for treating gas wells to enhance production.

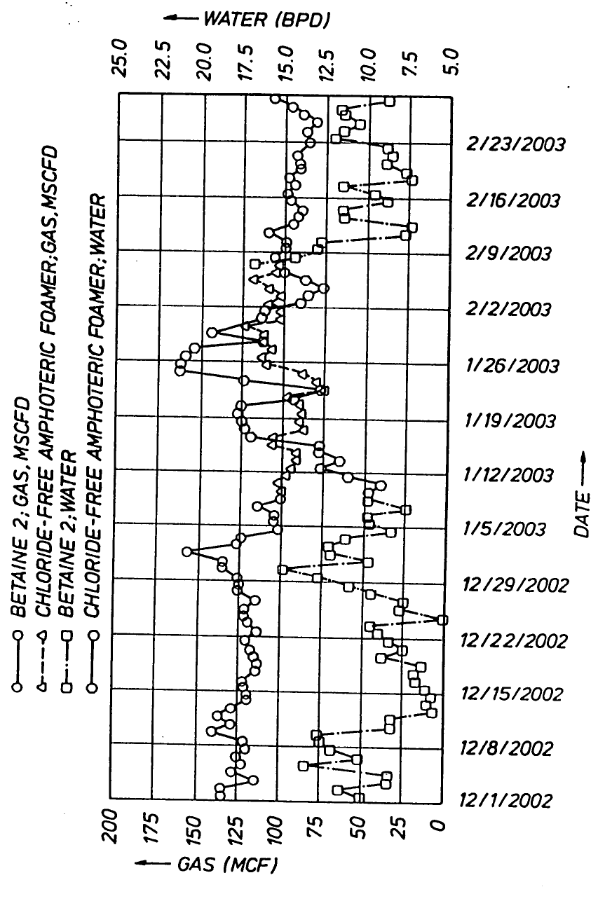

### BACKGROUND OF THE INVENTION

1. Field of the Invention

The present invention relates to non-corrosive amphoteric surfactants for use in the treatment of gas wells and, more particularly, to non-corrosive amphoteric surfactants which can be used as forming agents to enhance production from gas wells.

2. Description of the Prior Art

In natural gas wells, over time, gas production slows as the reservoir gas pressure decreases. A frequent cause of this loss of gas production is liquid loading that occurs when water and condensate from the formation flow into the well. As these liquids accumulate in the well, the gas to liquid ratio decreases, flowing velocity decreases, and the hydrostatic pressure in the well bore increases. With the increase in hydrostatic pressure, gas production may decrease and stop altogether. It has been estimated that liquid loading may affect gas production for 75 percent of a well's total production life. With the continuing demand for natural gas, treatment of wells to reduce the effects of liquid loading and enhance the recovery of natural gas is becoming increasingly important.

Several techniques have been proposed to reduce the impact of liquid loading in gas wells. Frequently, several technologies are combined to obtain optimal results. Commonly used technologies include periodic blowdowns (“stop cocking”); the use of rod/beam pumps; gas lift; plunger lift; automatic casing swabs; timers for intermittent lift; installation of smaller bore production tubing such as coil tubing or velocity strings, and the application of foaming agents.

Foaming agents are frequently chosen to increase liquid unloading from gas wells. Foaming techniques are not capital intensive and can be done in batch or continuous fashion. Several types of chemical agents have been used in these foaming applications including amphoteric, anionic, cationic and nonionic surfactants. In particular, amphoteric surfactants have several properties that make them particularly useful as compared with other surfactants. For one, they are easily made from readily available raw materials, they can be used in wells with low to high chloride concentrations and they are effective in wells containing condensate.

Foaming agents function by lowering surface tension thereby creating stable water/gas or water/gas/condensate foams. As is well known the energy required to lift foam from a well is substantially lower than the energy required to lift liquids such as water and/or hydrocarbon condensates.

Foaming agents can be applied in several ways including batch treatments using liquids and/or solid foamers and continuous applications via the casing/tubing annulus or via capillary strings.

Capillary strings with variable diameters can be placed directly in the production tubing. Typically, the capillary string is secured using a packoff assembly and tubing clamp. The use of capillary strings has the advantage that the chemical can be applied at the point where it will exert the greatest effect.

There have been cases when the use of amphoteric surfactants as foaming agents has been associated with corrosion failures in capillary strings. More specifically, cracking and pitting corrosion mechanisms have been identified in the failures even though the capillary strings are made from a CRA (corrosion resistant alloy). It has been suggested that some of the corrosion can be attributed to the chloride content of the commonly used amphoteric foaming agents. In this regard, most amphoteric surfactants are made from a process that generates 2 to 10 percent sodium chloride as a reaction product. Further, additional chloride may be added to reduce the viscosity of solutions of the amphoteric foamers.

### SUMMARY OF THE INVENTION

In a preferred aspect, the present invention provides a method for the preparation of chloride-free amphoteric surfactants which can be used, inter alia, as foaming agents. Amphoteric surfactants produced according to the process of the present invention show little or no corrosivity with respect to the alloys commonly used to manufacture capillary strings. Indeed, pitting and crevice corrosion attacks have not been observed on capillary string metals exposed to the chloride-free amphoteric surfactants of the present invention.

In another preferred aspect of the present invention, there is provided a method of treating a gas well to reduce liquid loading wherein a foaming agent comprising an amphoteric surfactant produced according to the present invention in an aqueous mixture is introduced into the well bore.

In yet another preferred aspect of the present invention there is provided a chloride free amphoteric surfactant that can be used in gas well treatments.

### BRIEF DESCRIPTION OF THE DRAWINGS

The single fixture is a graph comparing the use of an amphoteric surfactant in accordance with the present invention and a typical prior art amphoteric surfactant containing chloride.

### DETAILED DESCRIPTION OF PREFERRED EMBODIMENTS

Synthesis

According to one aspect of the present invention, fatty amines are reacted with unsaturated compounds containing a carbonyl group (carbonyl compound) that is adjacent to the double bond, e.g., carboxylic acids, esters and other derivatives. Non-limiting examples include acrylic acid, methacrylic acid, methyl acrylate, methyl methacrylate, etc. The resulting amine is quaternized and; optionally, can be converted to a salt that forms a highly effective foaming agent. Foaming agents of the present invention are typically made in a mixed solvent system that may contain water, alcohols, glycols, glycol ethers or the like. The reaction may be conducted with or without a catalyst, alkali metal hydroxides being the preferred catalysts if a catalyst is used. The reaction between the amine and the carbonyl compound is commonly referred to as a Michael Addition and is shown schematically below. 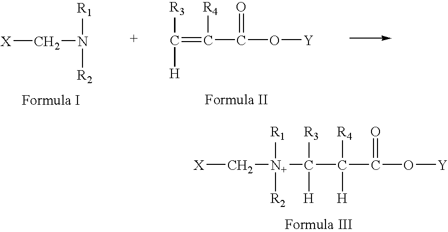
wherein X- is a hydrocarbyl group containing from 2 to 36 carbon atoms, preferably 2 to 20 carbon atoms, and can be optionally substituted with amido, amino, ester groups and the like. R₁, R₂, R₃, and R₄ can independently be hydrogen or an alkyl group containing from 1 to 4 carbon atoms and Y is hydrogen or an alkyl group containing from 1 to 4 carbon atoms which may be substituted with other groups as noted above with respect to the X grouping.

The reaction can be conducted over wide temperature and pressure ranges, temperatures of between 10° and 150° C. being operable. Although it is not necessary to conduct the reaction under pressure, this can be done if it is desired to operate at a lower temperature. Typically, the reaction is conducted with a nitrogen purge to prevent inadvertent oxidation of any of the reactants and/or products.

The amine can be conveniently derived from naturally occurring fatty acids by methods well known to those skilled in the art. Unlike prior art processes which also employ Michael Addition as a reaction sequence, the process of the present invention is conducted in the absence of chloride containing compounds.

The amphoteric surfactant represented by Formula III can be mixed with water in a weight ratio of surfactant to water of from 1:46 to 1:10.

Foam Testing

Foaming agents such as the ones produced according to the process of the present invention can be evaluated using a variety of test methods. Generally, two methods have been used to evaluate the foaming efficacy of the chloride-free amphoteric surfactant: blender tests (ASTM D-3519-88) and a modified ASTM-D-892 test method.

1. Blender Test, ASTM D-3519-88

In the blender test a set volume of brine (produced or synthetic) is treated with a foaming agent. The typical volume used in the test is 100-200 mL. The treated brine is subjected to a high shear rate for 30-60 sec. A shear rate of 3,000-14,000 rpm is recommended. After 30-60 sec. the blender is turned off and the foam height is measured in mm or mL. The time to defoam one-half of the initial charge is recorded as the foam half-life. Typical test times are 5-10 minutes.

In some cases the effect of adding hydrocarbon condensates is measured using the blender test. In these tests a produced or synthetic hydrocarbon is added to the brine before shearing. After shearing, the foam height and foam half-life are calculated as described above.

2. Modified ASTM D-892 Test

In the modified D-892 Test, gas is used to create foam instead of mechanical shearing. To simulate gas production a gas rich in methane is used instead of an inert gas or air. The flow rate of the methane-rich gas is typically maintained at a rate of 0.25 to 1.0 L/min using a calibrated flowmeter. Tests can be conducted in a cylinder with a capacity of 700 or 1,000 mL. Test volumes vary with 100 mL of brine being a common charge. For tests at temperatures above or below ambient temperature, ±25 C, a jacketed column can be used. The temperature inside the jacketed column can be maintained at 0-100C ±1 C using a circulating water bath such as a Fisher Scientific Isotemp 910.

In the modified D-892 test, foam height in mL is recorded at various intervals. The column is also fitted with an overflow device that allows for the determination of liquid overflow during a test. Liquid overflow is determined in mL at the end of the test period. The typical test time is 10 to 30 min. At the end of the test the foam height after overflow and the foam half-life are determined.

Corrosion Testing

Two methods are used for determining the corrosion rates of capillary strings made from different metallurgies. The first method determines the corrosion rate of a coupon in contact with the foaming agent at elevated temperatures in a sealed glass test tube. The second method involves exposure of a coupon to oxygen and the foaming agent in an autoclave. In both tests, the coupons are removed and examined with a microscope. General and pitting corrosion rates are determined by weight loss and pit depth, respectively. Typical corrosion rates for amphoteric surfactants containing chloride have been determined to vary widely with the mechanism of attack. Localized corrosion rates due to pitting may be very high—greater than 100 mpy (2.5 mm/yr). General corrosion rates are very low—less than 1 mpy (0.025 mm/yr).

1. Corrosion Rate in Constant Temperature Bath

Coupons of various metallurgies—e.g., SS 316, Duplex 2205, 1-825, and various shapes are prepared and cleaned. An acceptable cleaning procedure includes sandblasting, ultrasonic washing with an aromatic solvent (toluene, xylene) and two washes in acetone. Cleaned and dried coupons are measured, weighed to the nearest 0.0001 mg and stored in a desiccator until needed.

Prepared coupons are introduced to a thick-wall glass test tube fitted with a resin cap. Foaming agents are introduced into the test cells and the test tube assembly is sealed. Test tubes containing the coupon and foaming agent are then immersed in a sand bath at a thermostatically controlled temperature. The temperature limit typically used for the constant temperature bath is 165 C.

Tests are normally run for three days and the coupons may be observed several times during the test period. At the end of the test the coupons are removed, cleaned and re-weighed to the nearest 0.0001 mg. Cleaned coupons are examined microscopically for evidence of corrosion: general attack, pitting attack, edge attack and crevice corrosion. General corrosion rates are determined from differences in the weight of the coupon. The extent of pitting attack can be estimated from the number of pits and the average depth of the pits.

2. Corrosion Rate Determination in Stirred Autoclave

A second method for determining the corrosion rate of capillary string metals involves the use of a stirred autoclave. Coupons of various metallurgies are cleaned, measured and weighed as noted above.

Prepared coupons are inserted into autoclaves with a capacity of two to five L. An autoclave with a capacity of three L is recommended. Approximately 1.4 L of a foaming agent is added to the autoclave. After the coupon and foaming agent have been charged, the autoclave is closed and charged with breathing air and an inert gas. Prior to heating to the test temperature, the solutions are stirred. At this time dissolved oxygen readings are obtained using a suitable dissolved oxygen meter and an intrusive probe. Solutions are then heated to the test temperature and held at the test temperature for three to fourteen days. Typical test temperatures range from 100 to 200 C.

A typical protocol for the three to fourteen day test uses the following discreet steps:

a. heat to test temperature

b. cool to room temperature overnight

c. stir for five minutes, record dissolved oxygen content; if the oxygen level falls more than 50% recharge with air

d. remove the coupons and determine the nature of the corrosion mechanism and the corrosion rate(s)

Synthesis Example

In an appropriate container fitted with a reflux condenser, one mole of a fatty amino amine, cocoamidopropyl N,N dimethylamine, is mixed with a 50:50 mixture of water and the monobutyl ether of ethylene glycol (butyl cellosolve). The resulting mixture is heated to 100 C. One mole of a acrylic acid is added while stirring the mixture. Temperature is thermostatically controlled during the addition at 100 C. When addition is complete, the reaction mixture is stirred at 100 C for an additional 4 hours. Upon completion of the reaction, the resulting product was tested for foaming quality and corrosivity.

Foaming Efficacy

Foaming qualities of the chloride-free amphoteric surfactant were compared to an amphoteric surfactant containing chloride. The chloride containing surfactant was the betaine of cocoamidopropyl N,N dimethylamine. The betaine was obtained from a commercial manufacturer and contained 2-10% by weight chloride as NaCl.

Foam qualities were determined by injecting 1,000 ppmv (vol/vol) of foaming agent in NACE brine and in tap water. The typical chemical makeup of NACE brine and tap water are found in Table 1.

TABLE 1
Brine Make-up for Blender Foaming Tests

| Chemical Component | Tap Water | NACE Brine |
|---|---|---|
| Calcium mg/L | 14 | 829 |
| Magnesium mg/L | 5 | 222 |
| Barium mg/L | 0 | 0 |
| Strontium mg/L | 0 | 0 |
| Chloride mg/L | 60 | 60,470 |
| Sulfate mg/L | 292 | 0 |
| Bicarbonate mg/L | 395 | 0 |
| pH | 7.22 | |

Table 2 presents data from ASTM D 3519-88 tests. Results indicate that the betaine and chloride-free amphoteric surfactant produce stable foams.

TABLE 2
Foam Qualities in water using ASTM D 3519-88 Test Method

| Chemical | Foam Height, mL | Half-life in sec, t |
|---|---|---|
| | | Tap Water |
| Betaine | 850 | 281 |
| Amphoteric, chloride-free | 800 | 232 |
| | | NACE Brine |
| Betaine | 800 | 380 |
| Amphoteric, chloride free | 800 | 386 |

Foam qualities for the chloride-free surfactant and the betaine were also tested using the modified ASTM D-892 method. In this case, the gas used to generate foam is 100% methane and the flow rate through the 700-mL jacketed column was 1.0 L/min. Approximately 1,000 ppmv of foaming agent was used. The test brines were made from NaCl and contained 25,000 to 100,000 mg/L of chloride. Accumulated water in the foaming column at the end of the test was regarded as a negative indicator of foam stability. Lower values for the accumulated water indicated a more stable foam.

Table 3 presents test data from modified ASTM D-892 tests. Results for the chloride-free amphoteric surfactant are very similar to those obtained for the commercially available betaine.

TABLE 3
Foam Qualities in water containing NaCl using
the modified ASTM D-892 Test Method

| | Brine | Foam | Foam | |
|---|---|---|---|---|
| | Chloride | Height, | Height, | Returned |
| Product | conc. | 2 min, mL | 10 min, mL | Water, mL |
| Betaine | 25,000 | 690 | 620 | 6 |
| Amphoteric, | 25,000 | 650 | 630 | 4 |
| chloride-free | | | | |
| Betaine | 50,000 | 660 | 620 | 4 |
| Amphoteric, | 50,000 | 690 | 620 | 2 |
| chloride-free | | | | |
| Betaine | 100,000 | 680 | 635 | 5 |
| Amphoteric, | 100,000 | 650 | 620 | 4 |
| chloride-free | | | | |

A modified D-892 test was conducted using gas from a producing well. In these tests columns with capacities of 700-1,000 mL were used. The gas flow rate during the test was 1.0 L/min. Water and gas analyses for the produced water and gas are collected in Table 4.

TABLE 4
Water and Gas Analyses for Field Test

| Water Analysis | Gas Analysis |
|---|---|
| Specific Gravity: 1.080 | Carbon Dioxide: 0.80 mol % |
| pH: 5.94 | Methane: 92.16% |
| Calcium: 11,100 mg/L | Ethane: 4.54% |
| Magnesium: 135 mg/l | Propane: 1.10 |
| Barium: 689 mg/L | Hydrogen Sulfide: 23 ppm |
| Strontium: 1,590 mg/L | Nitrogen: 0.13% |
| Sodium: 27,700 mg/L | Heavier components: 1.27% |
| Potassium: 1.550 mg/L | |
| Iron: 5 mg/L | |
| Manganese: 7 mg/L | |
| Chloride: 80,000 mg/L | |
| Sulfate: 18 mg/L | |
| Bicarbonate (weak acids): 87 mg/L | |
| Dissolved carbon dioxide: 195 mg/L | |

Table 5 presents the results for modified D-892 tests conducted in the field using produced gas. Data for tests using 700 and 1,000 mL columns are included. Results from this test suggest that the chloride-free foamer performs similarly to the commercially available betaine.

TABLE 5
Field Foam Quality Tests using modified ASTM D-892 Test

| | Concen- | Foam | Foam | Water |
|---|---|---|---|---|
| | tration | Height | Height | Overflow, |
| Product | ppmv | 2 min, mL | 10 min, mL | mL |
| | | 700-mL column | | |
| Betaine | 1,000 | 700 | 350 | 69 |
| Amphoteric, | 1,000 | 700 | 400 | 68 |
| chloride-free | | | | |
| | | 1,000-mL column | | |
| Betaine | 1,000 | 1,100 | 850 | 52 |
| Amphoteric, | 1,000 | 1,100 | 800 | 48 |
| chloride-free | | | | |

Corrosivity Tests

Two sets of corrosivity tests were performed on a second commercially available betaine (Betaine 2) and the chloride-free amphoteric surfactant.

Table 6 presents the data from the constant temperature bath corrosion test. Results from this test clearly show that the general corrosion rates for the two products are similar. However, the pitting corrosion rates for Betaine 2, which contains 3-7% NaCl, are clearly higher. No evidence of pitting is seen on the coupons immersed in the chloride-free amphoteric surfactant. The maximum temperature, duration of exposure and metallurgy of the coupons are varied in the tests.

TABLE 6
Corrosion rates from the Constant Temperature Test

| | | Test Temp | General | |
|---|---|---|---|---|
| | Coupon | (C.)/Test | Corrosion | |
| Chemical | Metallurgy | Interval (hr) | Rate, mpy | Comments |
| Betaine 2 | SS 316 | 150/40 | 4.16 | Pitting |
| Amphoteric, | SS 316 | 150/40 | 0.41 | No Pitting |
| chloride-free | | | | |
| Betaine 2 | Duplex 2205 | 135/115 | 0.76 | Pitting |
| Amphoteric, | Duplex 2205 | 135/115 | 0.30 | No Pitting |
| chloride-free | | | | |

Table 7 presents the data from the stirred autoclave tests. In these tests coupons made from four different metals have been immersed in Betaine 2 and the chloride-free amphoteric surfactant for ten (10) days. Three different types of coupons were used: flat, U-bend and pieces of commercially available capillary strings. It is clear from these results that the chloride-free amphoteric surfactant is non-corrosive with respect to the alloys commonly used to manufacture capillary strings.

TABLE 7
Corrosion rates in the Stirred Autoclave Test

| | | | Localized | |
|---|---|---|---|---|
| | | | Corrosion | |
| | | Corrosion | Rate | Comments |
| | | Rate, weight | (Pitting), | Corrosion |
| Chemical | Alloy/Type | loss, mpy | mpy | Mechanism |
| Betaine 2 | 2507, flat | 0.35 | 95.51 | Some Pitting |
| Amphoteric | 2507, flat | 0.17 | 0.00 | No Pitting |
| Betaine 2 | 2205, flat | 0.50 | 136.45 | Pitting |
| Amphoteric | 2205, flat | 0.16 | 0.00 | No Pitting |
| Betaine 2 | SS 316, U- | 0.69 | 109.16 | Pitting, |
| | bend | | | Crevice |
| Amphoteric | SS 316, U- | 0.10 | 0.00 | No Pitting |
| | bend | | | |
| Betaine 2 | SS 316, flat | 0.58 | 136.45 | Pitting, |
| | | | | Crevice |
| Amphoteric | SS 316, flat | 0.13 | 0.00 | No Pitting |
| Betaine 2 | I-825, flat | 0.47 | 95.51 | Some Pitting, |
| | | | | Crevice |
| Amphoteric | I-825, flat | 0.08 | 0.00 | No Pitting |
| Betaine 2 | 2205, capillary | 0.73 | 81.87 | Pitting, |
| | | | | Crevice |
| Amphoteric | 2205, capillary | 0.20 | 0.00 | No Pitting |

With reference to the single figure, there is shown a comparison of the chloride free foamers of the present invention and a typical prior art amphoteric type surfactant (Betaine 2) contains chloride. It is to be noted that the Betaine 2 typically contains from 3 to 7 percent by weight sodium chloride as a result of the process by which it is produced. In conducting the comparative testing, an aqueous mixture containing one part of the chemical, e.g., Betaine 2 or the chloride free amphoteric surfactant produced according to the Synthesis Example were mixed with water in a weight ratio of one part chemical (surfactant) to seven parts water. The mixtures were injected through the production tubing of a gas well located in Texas, through a capillary tubing having a 0.25 inch OD at the same rate (gallons per day). Data was accumulated over an approximate three month period, the data consisting of gas production from the well and water production from the well. In conducting the test, Betaine 2 was initially used and gas and water production monitored from a period commencing on Dec. 1, 2002, and ending on Jan. 8, 2003. At that point, the chloride free amphoteric roamer of the present invention was then injected at the same rate as the Betaine 2 and gas and water production measured in the period spanning Jan. 9, 2003 until Feb. 7, 2003. At that point, a switch over was made back to the Betaine 2 roamer. The results are graphically depicted in the Figure.

Experiments Summary

As can be seen from the data above, amphoteric surfactants prepared according to the process of the present invention provide foaming efficiencies comparable to prior art amphoteric surfactants containing chlorides. In this regard see the results in Tables 2 and 3. Tests using field fluids and gas have confirmed this as shown by the data in Tables 5 and the Figure.

With respect to corrosion, and as can be seen in Tables 6 and 7, the chloride-free amphoteric surfactants prepared according to the process of the present invention display markedly reduced corrosion both with respect to general corrosion and localized corrosion (pitting). Indeed, as can be seen from the data in Table 7, the amphoteric surfactants of the present invention are non-corrosive with respect to the alloys commonly used to manufacture capillary strings.

1. A method of preparing a chloride free amphoteric surfactant comprising reacting an amine having the general formula: 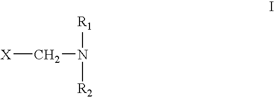
with a carbonyl compound having the formula: 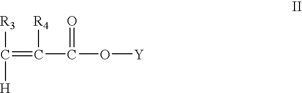
to produce an amphoteric surfactant having the formula: 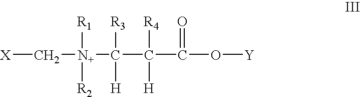
wherein X is a hydrocarbyl group containing from 2 to 36 carbon atoms, which can be optionally substituted with functional groups, R₁ R₂ R₃ and R₄ are independently hydrogen or a hydrocarbyl group containing from 1 to 4 carbon atoms and Y is hydrogen or a hydrocarbyl group containing from 1 to 4 carbon atoms wherein any of R₁ R₂ R₃ R₄ and Y can be optionally substituted with functional groups, and wherein said reaction is carried out in the substantial absence of any chloride containing compound.

2. The method of claim 1 wherein said reaction is conducted at a temperature of between 10° and 150° C.

3. The method of claim I wherein said reaction is conducted in a solvent system.

4. The method of claim 3 wherein said solvent system is selected from the group consisting of water, alcohols, glycols, glycol ethers and mixtures thereof.

5. The method of claim 1 wherein said reaction is conducted in the presence of an alkali metal hydroxide catalyst.

6. A chloride free amphoteric surfactant having the formula: 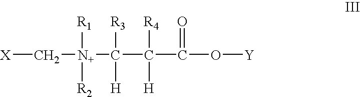
wherein X is a hydrocarbyl group containing from 2 to 36 carbon atoms, which can be optionally substituted with functional groups, R₁ R₂ R₃ and R₄ are independently hydrogen or a hydrocarbyl group containing from 1 to 4 carbon atoms and Y is hydrogen or a hydrocarbyl group containing from 1 to 4 carbon atoms wherein any of R₁ R₂ R₃ R₄ and Y can be optionally substituted with functional groups, said surfactant being free of any significant amount of chloride containing compounds.

7. A method of treating a gas well comprising:
introducing into said well an aqueous mixture comprising an effective amount of the composition of claim 6.

8. The method of claim 7 wherein the weight ratio of amphoteric surfactant to water in said aqueous mixture is from about 4 to 1 to about 10 to 1.
